(19) United States
(12) Patent Application Publication
(10) Pub. No.: US 2007/0196510 A1
(43) Pub. Date: Aug. 23, 2007

(54) Method for treating resistant hypertension

(76) Inventors: Michael J. Gerber, Denver, CO (US); Richard J. Gorczynski, Westminster, CO (US); Robert L. Roden, Broomfield, CO (US)

Correspondence Address: HARNESS, DICKEY, & PIERCE, P.L.C, 7700 BONHOMME, STE 400, ST. LOUIS, MO, 63105, US

(21) Appl. No.: 11/509,895

(22) Filed: Aug. 24, 2006

Related U.S. Application Data
(60) Provisional application No. 60/774,561, filed on Feb. 17, 2006.

(51) Int. Cl. A61K 33/24; A61K 31/554; A61K 31/549; A61K 31/455; A61K 31/4184; A61K 31/41; A61K 31/401

(52) U.S. Cl. 424/617; 514/355; 514/423; 514/381; 514/223.5; 514/471; 514/571; 514/412; 514/651; 514/278; 514/738; 514/411; 514/211.07

(57) ABSTRACT

A method is provided for lowering blood pressure in a patient having clinically diagnosed resistant hypertension. The method comprises administering darusentan to the patient adjunctively with a baseline antihypertensive regimen that comprises administration of at least one diuretic and at least two antihypertensive drugs selected from at least two of (a) ACE inhibitors and angiotensin II receptor blockers, (b) beta-adrenergic receptor blockers and (c) calcium channel blockers. The darusentan is orally administered at a dose and frequency effective, in combination with the baseline regimen, to provide a reduction of at least about 3 mmHg in one or more blood pressure parameters selected from trough sitting systolic, trough sitting diastolic, 24-hour ambulatory systolic, 24-hour ambulatory diastolic, maximum diurnal systolic and maximum diurnal diastolic blood pressures.

This application claims the benefit of U.S. provisional patent application Ser. No. 60/774,561, filed on Feb. 16, 2006, the entire disclosure of which is incorporated by reference herein. This application contains subject matter that is related to U.S. provisional patent application Ser. No. 60/774,560, Ser. No. 60/774,738, Ser. No. 60/774,739 and Ser. No. 60/774,884, each filed on Feb. 16, 2006, the entire disclosure of each of which is incorporated by reference herein.

### FIELD OF THE INVENTION

The present invention relates to methods and therapeutic combinations useful for lowering blood pressure.

### BACKGROUND OF THE INVENTION

Blood pressure control can often be achieved by antihypertensive therapy with one or more drugs. Despite a wide range of drugs available for antihypertensive therapy, a segment of the patient population continues to exhibit resistance to a baseline antihypertensive therapy with one or more drugs. A particularly challenging patient population has clinically diagnosed resistant hypertension. Resistant hypertension is defined by the Seventh Report of the Joint National Committee on Prevention, Detection, Evaluation, and Treatment of High Blood Pressure (JNC 7; Chobanian et al. (2003) Hypertension 42:1206-1252) as a failure to achieve goal blood pressure in patients who are adhering to full doses of an appropriate three-drug regimen that includes a diuretic. Further, resistant hypertension is diagnosed by many physicians on the basis of a patient's resistance to adequate, but less than full, doses of an appropriate three-drug regimen because of the risk or occurrence of adverse events associated with full doses. The terms “adequate” and “full” in the present context are defined hereinbelow.

According to JNC 7, a goal of systolic blood pressure (SBP)<140 mmHg and diastolic blood pressure (DBP)<90 mmHg is recommended for patients with hypertension and no other serious conditions. For patients with serious or compelling conditions such as diabetes and chronic kidney disease, JNC 7 recommends a goal of SBP<130 mmHg and DBP<80 mmHg. Despite intensive, multi-drug therapy, only about 50% of patients with diabetes or chronic kidney disease reach traditional blood pressure goals, with even fewer reaching the more stringent goals now recommended by JNC 7. Thus, resistant hypertension is particularly acute for segments of the population which exhibit diabetes or chronic kidney disease.

It should be noted that the British Hypertensive Society (BHD-IV; J. Human Hypertens. (2004) 18:139-185), the European Society of Hypertension/European Society of Cardiology (ESH/ESC; J. Hypertens. (2003) 21:1011-1053), and the World Health Organization/International Society of Hypertension (WHO/ISH; J. Hypertens. (2003) 21:1983-1992) guidelines propose similar but not identical blood pressure goals for nondiabetic and diabetic patients.

Darusentan is an endothelin-A (ET_{A}) selective receptor antagonist which has been used to treat moderate hypertension. Endothelin is a small peptide hormone that is believed to play a critical role in control of blood flow and cell growth. Elevated endothelin blood levels are associated with several cardiovascular disease conditions, including pulmonary arterial hypertension, chronic renal disease, coronary artery disease, hypertension, and chronic heart failure. Endothelin is a potent vasoconstrictor, triggering contraction through endothelin-receptor mediated signaling pathways.

Nakov et al. (2002) Am. J. Hypertens. 15:583-589 described a 392-patient study in which moderate hypertension was treated with darusentan at 10 to 100 mg/day. Exclusion criteria included concomitant medication with other antihypertensive drugs. Darusentan was reported to significantly reduce SBP and DBP by comparison with placebo.

German Patent No. DE 19744799 of Knoll mentions, in the abstract thereof, combinations of an endothelin antagonist, such as darusentan, and a diuretic said to show synergistic activity in treatment of hypertension, coronary artery disease, cardiac or renal insufficiency, renal or myocardial ischemia, subarachnoid hemorrhage, Raynaud's disease and peripheral arterial occlusion.

U.S. Pat. No. 6,352,992 to Kirchengast et al. proposes pharmaceutical combination preparations comprising a beta-receptor blocker and an endothelin antagonist for treatment of vasoconstrictive disorders. Among endothelin antagonists mentioned is darusentan.

German Patent No. DE 19743142 of Knoll proposes combinations of an endothelin antagonist, such as darusentan, and a calcium antagonist for treatment of cardiovascular disorders such as pulmonary hypertension and renal and myocardial ischemia.

U.S. Pat. No. 6,329,384 to Munter et al. proposes combinations of endothelin antagonists, such as darusentan, and renin-angiotensin system inhibitors, in particular angiotensin II antagonists and angiotensin converting enzyme (ACE) inhibitors for treatment of vasoconstrictive disorders such as hypertension, heart failure, ischemia or vasospasms.

German Patent No. DE 19743140 of Knoll proposes combinations of an endothelin antagonist, such as darusentan, and a vasodilator for treatment of cardiovascular disorders such as pulmonary hypertension, renal or myocardial ischemia, subarachnoid hemorrhage, Raynaud's disease, and peripheral arterial occlusion.

International Patent Publication No. WO 2004/082637 of Pharmacia proposes combinations of an aldosterone receptor antagonist with an endothelin receptor antagonist and/or an endothelin converting enzyme inhibitor, compositions thereof, and therapeutic methods for use in treatment of pathological conditions such as hypertension, cardiovascular disease and renal dysfunction.

Improved drug therapies for treatment of patients exhibiting resistance to a baseline antihypertensive therapy with one or more drugs, and especially patients having medically diagnosed resistant hypertension, would be highly desirable. Resistant hypertension is increasing in prevalence due to a variety of contributing factors including an aging population, obesity, patient noncompliance, and the effects of target-organ disease. Thus, a focus of current treatment of resistant hypertension is to identify and eliminate contributing factors. Despite reducing contributing factors, a substantial proportion of patients with resistant hypertension fail to achieve blood pressure goals.

A Myogen, Inc. news release dated Jul. 15, 2004 (http://investor.myogen.com/phoenix.zhtml?c=135160&p=irol-newsArticle&ID=759464&highlight=), later reported in Heart Disease Weekly, Aug. 15, 2004, p. 113, announced initiation of a clinical trial to evaluate safety and efficacy of darusentan in patients with resistant systolic hypertension.

### SUMMARY OF THE INVENTION

There is now provided a method for lowering blood pressure in a patient having clinically diagnosed resistant hypertension, the method comprising administering darusentan to the patient adjunctively with a baseline antihypertensive regimen that comprises administration of at least one diuretic and at least two antihypertensive drugs selected from at least two of (a) ACE inhibitors and angiotensin II receptor blockers, (b) beta-adrenergic receptor blockers and (c) calcium channel blockers; wherein the darusentan is orally administered at a dose and frequency effective, in combination with said regimen, to provide a reduction of at least about 3 mmHg in one or more blood pressure parameters selected from trough sitting systolic, trough sitting diastolic, 24-hour ambulatory systolic, 24-hour ambulatory diastolic, maximum diurnal systolic and maximum diurnal diastolic blood pressures.

Other embodiments, including particular aspects of the embodiments summarized above, will be evident from the detailed description that follows.

### BRIEF DESCRIPTION OF THE DRAWINGS

FIG. 1 is a schematic diagram of the clinical study described in Example 1 herein.

FIG. 2 is a flow diagram of patient disposition in the study described in Example 1 herein.

FIG. 3 is a graphical representation of systolic blood pressure (SBP) results obtained in the study described in Example 1 herein.

FIG. 4 is a graphical representation of diastolic blood pressure (DBP) results obtained in the study described in Example 1 herein.

FIG. 5 is a graphical representation of systolic ambulatory blood pressure (ABP) results over a 24-hour period as described in Example 3 herein.

### DETAILED DESCRIPTION

In various aspects of the invention, a method is provided for lowering blood pressure in a patient exhibiting resistance to a baseline antihypertensive therapy with one or more drugs.

Any one or more measures of blood pressure can be lowered by a method as described herein, including SBP and/or DBP as determined, for example, by sphygmomanometry. SBP and/or DBP can be measured, for example, in a sitting or ambulatory patient.

A “trough sitting” SBP or DBP is measured at a time point when serum concentration of a drug or drugs administered according to a method of the invention is expected to be at or close to its lowest in a treatment cycle, typically just before administration of a further dose. Illustratively, where the drug or drugs are administered once a day at a particular time, for example around 8 am, trough sitting systolic or diastolic blood pressure can be measured at that time, immediately before the daily administration. It is generally preferred to measure trough sitting SBP or DBP at around the same time of day for each such measurement, to minimize variation due to the natural diurnal blood pressure cycle.

A “24-hour ambulatory” SBP or DBP is an average of measurements taken repeatedly in the course of a 24-hour period, in an ambulatory patient.

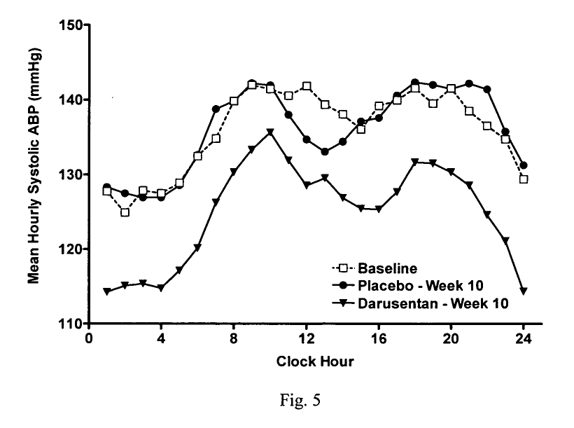

A “maximum diurnal” SBP or DBP is a measure of highest SBP or DBP recorded in a 24-hour period, and often reflects the peak of the natural diurnal blood pressure cycle, typically occurring in the morning, for example between about 5 am and about 11 am. Commonly, a second peak occurs in the evening, for example between about 5 pm and 10 pm. Such a bimodal waveform diurnal ABP pattern may be especially characteristic of resistant hypertension. An illustrative diurnal SBP cycle, as affected by treatment with darusentan according to a method of the invention, is shown in FIG. 5.

A common feature of resistant hypertension is a nighttime (defined herein as 2200 to 0600) mean systolic ABP that is less than about 10% lower than the daytime (defined herein as 0600 to 2200) mean systolic ABP. The parameter herein termed “day/night ABP ratio” expressed as a percentage is calculated as (daytime mean−nighttime mean)/daytime mean×100. A diurnal ABP pattern having a day/night ABP ratio of less than about 10% is sometimes referred to as a “non-dipping ABP”.

As indicated above, the patient receiving blood pressure lowering (antihypertensive) therapy according to a method of the invention can be a patient exhibiting resistance to a baseline antihypertensive therapy with one or more drugs. A “baseline antihypertensive therapy” herein means a therapeutic regimen comprising administration of one or more drugs, not including darusentan, with an objective (which can be the primary objective or a secondary objective of the regimen) of lowering blood pressure in a hypertensive patient. Each drug according to the regimen is administered at least at a dose considered by an attending physician to be adequate for treatment of hypertension, taking into account the particular patient's medical condition and tolerance for the drug without unacceptable adverse side-effects. An “adequate” dose as prescribed by the physician can be less than or equal to a full dose of the drug. A “full” dose is the lowest of (a) the highest dose of the drug labeled for a hypertension indication; (b) the highest usual dose of the drug prescribed according to JNC 7, BHD-IV, ESH/ESC or WHO/ISH guidelines; or (c) the highest tolerated dose of the drug in the particular patient.

A baseline antihypertensive therapy illustratively comprises administering one or more diuretics and/or one or more antihypertensive drugs selected from (a) angiotensin converting enzyme inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, and (g) aldosterone receptor antagonists. Optionally drugs of still further classes can be included in the baseline therapy, for example to address secondary conditions occurring in a hypertensive patient or side-effects of one or more of the diuretic or antihypertensive drugs.

A patient who is “resistant” to a baseline antihypertensive therapy is one in whom hypertension is failing to respond adequately or at all to the baseline therapy. Typically, the patient receiving the baseline therapy is failing to reach an established blood pressure goal, as set forth for U.S. patients, for example, in JNC 7 or comparable standards in other countries (e.g., BHD-IV, ESH/ESC or WHO/ISH guidelines). Illustratively, the JNC 7 goal for SBP is <140 mmHg and for DBP<90 mmHg, or for a patient having a complicating condition such as diabetes and/or chronic kidney disease, <130 mmHg SBP and <80 mmHg DBP.

Patients resistant to a baseline antihypertensive therapy, especially such a therapy involving a plurality of drugs, clearly represent a very challenging population for treatment. Typically in such patients, increasing dosages of the baseline therapy are not an option because of resulting adverse side effects; furthermore this approach is often ineffective in providing a desired lowering of blood pressure.

Surprisingly, it has now been found in a clinical study involving such a challenging population of patients, that a clinically meaningful lowering of blood pressure can be obtained by use of the selective ETA receptor antagonist darusentan ((+)-(S)-2-[(4,6-dimethoxy-2-pyrimidinyl)oxy]-3-methoxy-3,3-diphenylpropionic acid). A reduction of at least about 3 mmHg in any blood pressure parameter can be considered clinically meaningful. Accordingly, in one embodiment of the present invention, a method for lowering blood pressure in a patient exhibiting resistance to a baseline antihypertensive therapy comprises administering to the patient darusentan at a dose and frequency effective to provide a reduction of at least about 3 mmHg in trough sitting SBP and/or DBP, 24-hour ambulatory SBP and/or DBP, and/or maximum diurnal SBP and/or DBP.

Further, in patients exhibiting resistance to a baseline antihypertensive therapy with one or more drugs, administration of darusentan adjunctively with these same drugs is surprisingly well tolerated. Accordingly, in another embodiment of the present invention, a method for lowering blood pressure in a patient in a patient exhibiting resistance to a baseline antihypertensive therapy with one or more drugs comprises administering darusentan to the patient adjunctively with said one or more drugs.

In the clinical study mentioned above (and described more fully in the Example hereinbelow) where a clinically meaningful reduction in blood pressure was surprisingly obtained in a patient population exhibiting resistance to a baseline antihypertensive therapy, darusentan was administered orally. Accordingly, in yet another embodiment of the present invention, a method for lowering blood pressure in a patient exhibiting resistance to a baseline antihypertensive therapy comprises orally administering darusentan to the patient.

While in certain embodiments the darusentan can be administered alone, i.e., in monotherapy, it is contemplated that in most cases combination therapy, for example but not necessarily with one or more drugs of the baseline therapy to which the patient has proved resistant, will be desirable. However, a benefit of the administration of darusentan can be that, at least in some circumstances, it can permit dose reduction, or even elimination, of at least one of the drugs in the baseline therapy.

Particularly when used at a full dose, many baseline antihypertensive therapy drugs can have undesirable, in some cases clinically unacceptable or even dangerous, adverse side effects.

For example, especially at full doses, potassium-sparing diuretic drugs can be associated with increased risk of hyperkalemia and related disorders. Overuse of loop diuretics can cause depletion of sodium resulting in hyponatremia and/or extracellular fluid volume depletion associated with hypotension, reduced GRF, circulatory collapse, and thromboembolic episodes. Further, loop diuretics can cause ototoxicity that results in tinnitus, hearing impairment, deafness and/or vertigo. Thiazide diuretics, similarly to loop diuretics, can have adverse effects related to abnormalities of fluid and electrolyte balance. Such adverse events include extracellular volume depletion, hypotension, hypokalemia, hyponatremia, hypochloremia, metabolic alkalosis, hypomagnesemia, hypercalcemia and hyperuricemia. Thiazide diuretics can also decrease glucose tolerance, and increase plasma levels of LDL (low density lipoprotein) cholesterol, total cholesterol, and total triglycerides.

Angiotensin converting enzyme (ACE) inhibitors are associated with cough and increased risk of angioedema. Beta-adrenergic receptor blockers are associated with increased risk of bronchospasm, bradycardia, heart block, excess negative inotropic effect, peripheral arterial insufficiency and sometimes male impotence. Calcium channel blockers are associated with increased risk of lower limb edema. Further information on adverse events associated with antihypertensive drugs can be found, for example, in standard reference works such as Goodman & Gilman's The Pharmaceutical Basis of Therapeutics, 13th ed.

In situations such as those outlined immediately above, dose reduction or elimination of a baseline therapy drug permitted by use of darusentan can result in a reduced risk or incidence of adverse events by comparison with the baseline therapy alone without such dose reduction or elimination.

“Adjunctive” administration of darusentan herein means that the darusentan is administered concomitantly with a baseline hypertensive therapy as defined above, with or without dose reduction of one or more drugs in the baseline therapy. For example, darusentan can be administered adjunctively with an adequate to full dose of each of the drugs in the baseline therapy. In adjunctive therapy, the dose and frequency of darusentan administration is, in one embodiment, effective in combination with the baseline therapy to provide a reduction of at least about 3 mmHg in trough sitting SBP and/or DBP, 24-hour ambulatory SBP and/or DBP, and/or maximum diurnal SBP and/or DBP.

A method of the present invention is especially beneficial where the patient has clinically diagnosed resistant hypertension. By definition herein, in general accordance with JNC 7, such a patient exhibits resistance to an antihypertensive regimen of at least three drugs including a diuretic. In one embodiment, the patient having resistant hypertension exhibits resistance to a baseline antihypertensive therapy that comprises at least the following:
(1) one or more diuretics; and
(2) two or more antihypertensive drugs, selected from at least two of the following classes:
   (a) ACE inhibitors and angiotensin II receptor blockers;
   (b) beta-adrenergic receptor blockers; and
   (c) calcium channel blockers.

In some cases, the patient is resistant to an even more comprehensive baseline therapy, further comprising, for example, one or more direct vasodilators, alpha-1-adrenergic blockers, central alpha-2-adrenergic agonists or other centrally acting antihypertensive drugs, and/or aldosterone receptor antagonists.

In one embodiment, the patient has medically diagnosed resistant systolic hypertension, and the dose and frequency of darusentan administration is effective in combination with the baseline therapy to provide a reduction of at least about 3 mmHg in one or more of trough sitting, 24-hour ambulatory and maximum diurnal SBP.

In a more particular embodiment, the at least about 3 mmHg reduction is observed in trough sitting SBP, and at least comparable reductions can be, but are not necessarily, observable in 24-hour ambulatory and/or maximum diurnal SBP. In some cases the method is effective to provide a greater reduction in trough sitting SBP, for example at least about 5 mmHg, at least about 7 mmHg or at least about 10 mmHg.

The method can increase the likelihood of a patient achieving SBP goal, for example a JNC 7, BHD-IV, ESH/ESC or WHO/ISH goal for SBP. Thus in a particular embodiment, a JNC 7 goal for SBP is achieved, for example a trough sitting or 24-hour ambulatory SBP of <140 mmHg or, in the case of a patient with diabetes or chronic kidney disease, <130 mmHg.

In another embodiment, the patient has medically diagnosed resistant diastolic hypertension, and the dose and frequency of darusentan administration is effective in combination with the baseline therapy to provide a reduction of at least about 3 mmHg in one or more of trough sitting, 24-hour ambulatory and maximum diurnal DBP.

In a more particular embodiment, the at least about 3 mmHg reduction is observed in trough sitting DBP, and at least comparable reductions can be, but are not necessarily, observable in 24-hour ambulatory and/or maximum diurnal DBP. In some cases the method is effective to provide a greater reduction in trough sitting DBP, for example at least about 5 mmHg, at least about 7 mmHg or at least about 10 mmHg.

The method can increase the likelihood of a patient achieving DBP goal, for example a JNC 7, BHD-IV, ESH/ESC or WHO/ISH goal for DBP. In a particular embodiment, a JNC 7 goal for DBP is achieved, for example a trough sitting or 24-hour ambulatory DBP of <90 mmHg or, in the case of a patient with diabetes or chronic kidney disease, <80 mmHg.

In yet another embodiment, the method is effective to increase day/night ABP ratio, for example from a baseline below about 10% to greater than 10%. Day/night ABP ratio can illustratively be increased by at least about 2, for example at least about 3 or at least about 5 percentage points.

In yet another embodiment, the method is effective to lower blood pressure at all times in a diurnal blood pressure cycle, for example as measured by ABP monitoring at a suitable interval, e.g., hourly. According to this embodiment, the diurnal blood pressure cycle can exhibit a bimodal waveform pattern both at baseline and when treated with darusentan according to the present method, but darusentan treatment shifts the waveform pattern downward as illustrated, for example, in FIG. 5.

Because of the particular criticality of controlling blood pressure in patients with complicating conditions such as diabetes and/or chronic kidney disease, and the greater difficulty of lowering blood pressure to the lower levels consistent with good management of these conditions, a method of the invention can be especially beneficial for such patients.

While in one embodiment, as mentioned above, the darusentan is administered orally, the invention is not limited to any route of administration of the darusentan, so long as the route selected results in effective delivery of the drug so that the stated benefits are obtainable. Thus administration of the darusentan can illustratively be parenteral (e.g., intravenous, intraperitoneal, subcutaneous or intradermal), transdermal, transmucosal (e.g., buccal, sublingual or intranasal), intraocular, intrapulmonary (e.g., by inhalation) or rectal. Most conveniently for the majority of patients, however, the darusentan is administered orally, i.e., per os (p.o.). Any suitable orally deliverable dosage form can be used for the darusentan, including without limitation tablets, capsules (solid- or liquid-filled), powders, granules, syrups and other liquids, etc.

For oral administration, any dose of darusentan that is therapeutically effective, up to a maximum that is tolerated by the patient without unacceptable adverse side effects, can be administered. For most patients, such a dose is likely to be about 1 to about 600 mg/day, for example about 5 to about 450 mg/day or about 10 to about 300 mg/day. Higher or lower doses can be useful in specific circumstances.

The prescribed daily dosage amount can be administered in any suitable number of individual doses, for example four times, three times, twice or once a day. With a dosage form having appropriate controlled release properties, a lower frequency of administration may be possible, for example once every two days, once a week, etc.

Most antihypertensive medicines are suitable for once a day administration, and this is true also of darusentan. Thus, particularly where darusentan is being administered in adjunctive therapy with one or more other antihypertensive drugs, it is generally most convenient to administer the darusentan once a day in a dose as indicated above.

Most typically, where the patient has medically diagnosed resistant hypertension, the darusentan is administered adjunctively with (1) one or more diuretics; and (2) two or more antihypertensive drugs, selected from (a) ACE inhibitors and angiotensin II receptor blockers; (b) beta-adrenergic receptor blockers; and (c) calcium channel blockers. Each of these diuretic and antihypertensive drugs is typically administered at an adequate to full dose. One of skill in the art can readily identify a suitable dose for any particular diuretic or antihypertensive drug from publicly available information in printed or electronic form, for example on the internet.

Mention of a particular diuretic or antihypertensive drug in the present specification and claims will be understood, except where the context demands otherwise, to include pharmaceutically acceptable salts, esters, prodrugs, metabolites, racemates and enantiomers of the drug, to the extent that such salts, esters, prodrugs, metabolites, racemates or enantiomers exist and are therapeutically effective.

Examples of drugs useful in combination or adjunctive therapy with darusentan or as a component of a baseline antihypertensive therapy are classified and presented in several lists below. Some drugs are active at more than one target; accordingly certain drugs may appear in more than one list. Use of any listed drug in a combination or adjunctive therapy of the invention is contemplated herein, independently of its mode of action.

A suitable diuretic can illustratively be selected from the following list.

Organomercurials
chlormerodrin
chlorothiazide
chlorthalidone
meralluride
mercaptomerin sodium
mercumatilin sodium
mercurous chloride
mersalyl

Purines
pamabrom
protheobromine
theobromine

Steroids
canrenone
oleandrin
spironolactone

Sulfonamide Derivatives
acetazolamide
ambuside
azosemide
bumetanide
butazolamide
chloraminophenamide
clofenamide
clopamide
clorexolone
disulfamide
ethoxzolamide
furosemide
mefruside
methazolamide
piretanide
torsemide
tripamide
xipamide

Thiazides and Analogs
althiazide
bendroflumethiazide
benzthiazide
benzylhydrochlorothiazide
buthiazide
chlorthalidone
cyclopenthiazide
cyclothiazide
ethiazide
fenquizone
hydrochlorothiazide
hydroflumethiazide
indapamide
methyclothiazide
metolazone
paraflutizide
polythiazide
quinethazone
teclothiazide
trichlormethiazide

Uracils
aminometradine

Unclassified
amiloride
Biogen BG 9719
chlorazanil
ethacrynic acid
etozolin
isosorbide
Kiowa Hakko KW 3902
mannitol
muzolimine
perhexiline
Sanofi-Aventis SR 121463
ticrynafen
triamterene
urea

In some embodiments, the diuretic if present comprises a thiazide or loop diuretic. Thiazide diuretics are generally not preferred where the patient has a complicating condition such as diabetes or chronic kidney disease, and in such situations a loop diuretic can be a better choice.

Particularly suitable thiazide diuretics include chlorothiazide, chlorthalidone, hydrochlorothiazide, indapamide, metolazone, polythiazide and combinations thereof. Particularly suitable loop diuretics include bumetanide, furosemide, torsemide and combinations thereof.

A suitable ACE inhibitor can illustratively be selected from the following list:
alacepril
benazepril
captopril
ceronapril
cilazapril
delapril
enalapril
enalaprilat
eosinopril
fosinopril
imidapril
lisinopril
moexipril
moveltipril
omapatrilat
perindopril
quinapril
ramipril
sampatrilat
spirapril
temocapril
trandolapril

Particularly suitable ACE inhibitors include benazepril, captopril, enalapril, fosinopril, lisinopril, moexipril, perindopril, quinapril, ramipril, trandolapril and combinations thereof.

A suitable angiotensin II receptor blocker can illustratively be selected from the following list:
candesartan
eprosartan
irbesartan
losartan
olmesartan
tasosartan
telmisartan
valsartan

A suitable beta-adrenergic receptor blocker can illustratively be selected from the following list:
AC 623
acebutolol
alprenolol
atenolol
amosulalol
arotinolol
atenolol
befunolol
betaxolol
bevantolol
bisoprolol
bopindolol
bucindolol
bucumolol
bufetolol
bufuralol
bunitrolol
bupranolol
butidrine hydrochloride
butofilolol
carazolol
carteolol
carvedilol
celiprolol
cetamolol
cloranolol
dilevalol
esmolol
indenolol
labetalol
landiolol
levobunolol
mepindolol
metipranolol
metoprolol
moprolol
nadolol
nadoxolol
nebivolol
nifenalol
nipradilol
oxprenolol
penbutolol
pindolol
practolol
pronethalol
propranolol
sotalol
sulfinalol
talinolol
tertatolol
tilisolol
timolol
toliprolol
xibenolol

Particularly suitable beta-adrenergic receptor blockers include acebutolol, atenolol, betaxolol, bisoprolol, carvedilol, labetalol, metoprolol, nadolol, penbutolol, pindolol, propranolol, timolol and combinations thereof.

A suitable calcium channel blocker can illustratively be selected from the following list:

Arylalkylamines
bepridil
clentiazem
diltiazem
fendiline
gallopamil
mibefradil
prenylamine
semotiadil
terodiline
verapamil

Dihydropyridine Derivatives
amlodipine
aranidipine
barnidipine
benidipine
cilnidipine
efonidipine
elgodipine
felodipine
isradipine
lacidipine
lercanidipine
manidipine
nicardipine
nifedipine
nilvadipine
nimodipine
nisoldipine
nitrendipine
NZ 105

Piperazine Derivatives
cinnarizine
dotarizine
flunarizine
lidoflazine
lomerizine

Unclassified
bencyclane
etafenone
fantofarone
monatepil
perhexiline

Particularly suitable calcium channel blockers include amlodipine, diltiazem, felodipine, isradipine, nicardipine, nifedipine, nisoldipine, verapamil and combinations thereof.

Optionally, one or more additional antihypertensive drugs can be administered. These can be selected, for example, from direct vasodilators, alpha-1-adrenergic receptor blockers, central alpha-2-adrenergic receptor agonists and other centrally acting antihypertensive drugs, and aldosterone receptor antagonists.

A suitable direct vasodilator can illustratively be selected from the following list:
amotriphene
benfurodil hemisuccinate
benziodarone
chloracizine
chromonar
clobenfurol
clonitrate
cloricromen
dilazep
droprenilamine
efloxate
erythrityl tetranitrate
etafenone
fendiline
hexestrol bis(β-diethylaminoethyl ether)
hexobendine
hydralazine
isosorbide dinitrate
isosorbide mononitrate
itramin tosylate
khellin
lidoflazine
mannitol hexanitrate
minoxidil
nitroglycerin
pentaerythritol tetranitrate
pentrinitrol
perhexiline
pimefylline
prenylamine
propatyl nitrate
trapidil
tricromyl
trimetazidine
trolnitrate phosphate
visnadine

Particularly suitable direct vasodilators include hydralazine, minoxidil and combinations thereof.

A suitable alpha-1-adrenergic receptor blocker can illustratively be selected from the following list:
amosulalol
arotinolol
carvedilol
dapiprazole
doxazosin
ergoloid mesylates
fenspiride
idazoxan
indoramin
labetalol
methyldopa
monatepil
naftopidil
nicergoline
prazosin
tamsulosin
terazosin
tolazoline
trimazosin
yohimbine

Particularly suitable alpha-1-adrenergic receptor blockers include carvedilol, doxazosin, labetalol, prazosin, terazosin and combinations thereof. It is noted that, of these, carvedilol and labetalol also function as beta-adrenergic receptor blockers.

A suitable central alpha-2-adrenergic receptor agonist or other centrally acting antihypertensive drug can illustratively be selected from the following list:
clonidine
guanabenz
guanadrel
guanfacine
methyldopa
moxonidine
reserpine

A suitable aldosterone receptor antagonist can illustratively be selected from the following list:
canrenone
eplerenone
spironolactone

Still further classes of drugs that can be useful in combination or adjunctive therapy with darusentan or in a baseline antihypertensive therapy include vasopeptidase inhibitors, NEP (neutral endopeptidase) inhibitors, prostanoids (particularly oral prostanoids), PDE5 (phosphodiesterase type 5) inhibitors, nitrosylated compounds and oral nitrates.

Illustrative vasopeptidase inhibitors include:
fasidotril
omapatrilat
sampatrilat

Illustrative NEP inhibitors, some of which are also ACE inhibitors, include:
candoxatril
CGS 26582
MDL 100173
omapatrilat
phosphoramidon
sinorphan
thiorphan
Z13752A

Illustrative prostanoids include:
beraprost
cicaprost
epoprostenol
iloprost
PGE₁
PGI₂ (prostacyclin)
NS-304
treprostinil

Illustrative PDE5 inhibitors include:
sildenafil
tadalafil
vardenafil

Other drugs that can be useful in combination or adjunctive therapy with darusentan or in a baseline antihypertensive therapy can illustratively be selected from the following unclassified list:
ajmaline
alfuzosin
Alteon ALT 711
γ-aminobutyric acid
atrial natriuretic peptide
azelnidipine
bethanidine
bietaserpine
bosentan
budralazine
bufeniode
bunazosin
cadralazine
carmoxirole
CD 3400
chlorisondamine chloride
cicletanine
ciclosidomine
clevidipine
debrisoquin
denitronipradilol
desacetylalacepril
deserpidine
diazoxide
dihydralazine
endralazine
fenoldopam
flosequinan
guanethidine
guanidine, N-cyano-N′-4-pyridinyl-N″-(1,2,2-trimethylpropyl)-, monohydrate
guanoxabenz
guanoxan
hexamethonium
ketanserin
LBI 45
levcromakalim
lofexidine
magnesiocard
mebutamate
mecamylamine
normopresil
2-oxazolamine, N-(dicyclopropylmethyl)-4,5-dihydro-, (2E)-2-butenedioate
pargyline
pempidine
pentamethonium bromide
pentolinium tartrate
pheniprazine
phentolamine
pildralazine
pinacidil
piperoxan
protoveratrines
3,5-pyridinedicarboxylic acid, 1,4-dihydro-2,6-dimethyl-4-(3-nitrophenyl)-, methyl 1-(phenylmethyl)-3-pyrrolidinyl ester
raubasine
rescimetol
rescinnamine
rilmenidine
saralasin
sodium niroprusside
syrosingopine
Takeda TAK 536
TBC 3711
tetrahydrolipstatin
1,4-thiazepine-4(5H)-acetic acid, 6-[[1-(ethoxycarbonyl)-3-phenylpropyl]amino]-tetrahydro-5-oxo-2-(2-thienyl)
tiamenidine
todralazine
tolonidine
trimethaphan camsylate
tyrosinase
urapidil
zofenopril

In one embodiment, the darusentan is administered concomitantly (e.g., in combination or adjunctive therapy) with one or more of
(a) a diuretic selected from the group consisting of chlorothiazide, chlorthalidone, hydrochlorothiazide, indapamide, metolazone, polythiazide, bumetanide, furosemide, torsemide and combinations thereof;
(b) an ACE inhibitor selected from the group consisting of benazepril, captopril, enalapril, fosinopril, lisinopril, moexipril, perindopril, quinapril, ramipril, trandolapril and combinations thereof, and/or an angiotensin II receptor blocker selected from the group consisting of candesartan, eprosartan, irbesartan, losartan, olmesartan, tasosartan, telmisartan, valsartan and combinations thereof;
(c) a beta-adrenergic receptor blocker selected from the group consisting of acebutolol, atenolol, betaxolol, bisoprolol, carvedilol, labetalol, metoprolol, nadolol, penbutolol, pindolol, propranolol, timolol and combinations thereof;
(d) a calcium channel blocker selected from the group consisting of amlodipine, diltiazem, felodipine, isradipine, nicardipine, nifedipine, nisoldipine, verapamil and combinations thereof;
(e) a direct vasodilator selected from the group consisting of hydralazine, minoxidil and combinations thereof;
(f) an alpha-1-adrenergic receptor blocker selected from the group consisting of carvedilol, doxazosin, labetalol, prazosin, terazosin and combinations thereof;
(g) a central alpha-2-adrenergic receptor agonist or other centrally acting drug selected from the group consisting of clonidine, guanabenz, guanadrel, guanfacine, methyldopa, moxonidine, reserpine and combinations thereof; and
(h) an aldosterone receptor antagonist selected from the group consisting of canrenone, eplerenone, spironolactone and combinations thereof.

More particularly, the darusentan can be administered in combination or adjunctive therapy with one or more of (a), (b), (c) and (d) above, optionally further with one or more of (e), (f), (g) and (h).

Still more particularly, the darusentan can be administered in combination or adjunctive therapy at least with (a) and any two of (b), (c) and (d).

As in the case of the darusentan, the one or more drugs constituting the baseline antihypertensive therapy and optionally administered in combination with the darusentan can be delivered by any suitable route of administration. Generally, such drugs are suitable for oral administration, and many are suitable for once a day oral administration. Thus in one embodiment at least one of the diuretic or antihypertensive drugs in the baseline therapy is orally administered once a day. In a particular embodiment, all drugs in the baseline therapy are orally administered once a day. According to this embodiment, it will generally be found convenient to administer all drugs in the regimen, i.e., the darusentan as well as the baseline therapy drugs, orally once a day.

Fixed-dose combinations of two or more drugs can be achieved in many cases by coformulation of the drugs in a single dosage unit such as a tablet or capsule. For example, coformulations of various drugs useful in a baseline antihypertensive therapy as defined herein are available, including:
amiloride+hydrochlorothiazide;
amlodipine+benazepril;
atenolol+chlorthalidone;
benazepril+hydrochlorothiazide;
bisoprolol+hydrochlorothiazide;
candesartan+hydrochlorothiazide;
captopril+hydrochlorothiazide;
enalapril+felodipine;
enalapril+hydrochlorothiazide;
eprosartan+hydrochlorothiazide;
fosinopril+hydrochlorothiazide;
irbesartan+hydrochlorothiazide;
lisinopril+hydrochlorothiazide;
losartan+hydrochlorothiazide;
methyldopa+hydrochlorothiazide;
metoprolol+hydrochlorothiazide;
moexipril+hydrochlorothiazide;
nadolol+hydrochlorothiazide;
olmesartan+hydrochlorothiazide;
propranolol+hydrochlorothiazide;
quinapril+hydrochlorothiazide;
reserpine+chlorothiazide;
reserpine+chlorthalidone;
reserpine+hydrochlorothiazide;
spironolactone+hydrochlorothiazide;
telmisartan+hydrochlorothiazide;
timolol+hydrochlorothiazide;
trandolapril+verapamil;
triamterine+hydrochlorothiazide; and
valsartan+hydrochlorothiazide.

When darusentan is used in adjunctive therapy with one or more baseline drugs, the darusentan and at least one baseline drug can be administered at different times or at about the same time (at exactly the same time or directly one after the other in any order). The darusentan and the at least one baseline drug can be formulated in one dosage form as a fixed-dose combination for administration at the same time, or in two or more separate dosage forms for administration at the same or different times.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, darusentan and at least one drug useful in combination or adjunctive therapy with the darusentan. In another example, the darusentan and the at least one drug useful in combination or adjunctive therapy with the darusentan are separately packaged and available for sale independently of one another, but are co-marketed or co-promoted for use according to the invention. The separate dosage forms can also be presented to a patient separately and independently, for use according to the invention.

A therapeutic combination comprising darusentan, at least one diuretic, and at least two antihypertensive drugs selected from at least two of (a) ACE inhibitors and angiotensin II receptor blockers, (b) beta-adrenergic receptor blockers and (c) calcium channel blockers is itself a further embodiment of the invention. Such a combination can have utility in a number of situations, not limited to methods described herein. However, a combination of this embodiment can be especially useful for lowering blood pressure in a patient exhibiting resistance to a baseline antihypertensive therapy with one or more drugs; for lowering blood pressure in a patient having diabetes and/or chronic kidney disease; for preventing one or more cardiovascular adverse events in a patient having medically diagnosed resistant hypertension; and/or for producing a beneficial effect on renal function in a patient having medically diagnosed resistant hypertension.

The at least one diuretic in the combination can illustratively be selected from those listed hereinabove. In particular embodiments the diuretic comprises a thiazide diuretic or a loop diuretic. Suitable ACE inhibitors, angiotensin II receptor blockers, beta-adrenergic receptor blockers and calcium channel blockers can illustratively be selected from those listed hereinabove. Optionally, the combination can further comprise one or more additional drugs selected from direct vasodilators, alpha-1-receptor blockers, central alpha-2-adrenergic receptor agonists and other centrally acting antihypertensive drugs, and aldosterone receptor antagonists. Suitable drugs of these classes are illustratively listed hereinabove.

In one embodiment, the combination comprises darusentan plus (a) and at least two of (b), (c) and (d) as described below:
(a) a diuretic selected from the group consisting of chlorothiazide, chlorthalidone, hydrochlorothiazide, indapamide, metolazone, polythiazide, bumetanide, furosemide, torsemide and combinations thereof;
(b) an angiotensin converting enzyme inhibitor selected from the group consisting of benazepril, captopril, enalapril, fosinopril, lisinopril, moexipril, perindopril, quinapril, ramipril, trandolapril and combinations thereof, and/or an angiotensin II receptor blocker selected from the group consisting of candesartan, eprosartan, irbesartan, losartan, olmesartan, tasosartan, telmisartan, valsartan and combinations thereof;
(c) a beta-adrenergic receptor blocker selected from the group consisting of acebutolol, atenolol, betaxolol, bisoprolol, carvedilol, labetalol, metoprolol, nadolol, penbutolol, pindolol, propranolol, timolol and combinations thereof;
(d) a calcium channel blocker selected from the group consisting of amlodipine, diltiazem, felodipine, isradipine, nicardipine, nifedipine, nisoldipine, verapamil and combinations thereof.

Typically at least the darusentan is provided in an orally deliverable formulation, for example a formulation adapted for oral delivery of a darusentan dose of about 1 to about 600 mg/day, e.g., about 10 to about 300 mg/day. The darusentan formulation can be adapted for any suitable frequency of administration, but in one embodiment is adapted for once a day oral administration.

In one embodiment at least one of the drugs other than darusentan in the combination is provided in an orally deliverable formulation; for example, each of the drugs can be so provided, and each of the drugs can be in a formulation adapted for once a day oral administration. Each of the drugs other than darusentan is typically present in the combination in an amount providing an adequate to full dose of the drug. One of skill in the art can readily identify a suitable dose for any particular drug from publicly available information in printed or electronic form, for example on the internet.

Any two or more drugs in the combination can optionally be coformulated to provide a fixed dose combination. For example, the darusentan can be coformulated with any one or more of the other drugs in the combination.

In a further embodiment, a method for lowering blood pressure in a patient having diabetes and/or chronic kidney disease comprises administering darusentan to the patient. The particular effectiveness of darusentan in lowering blood pressure is believed to be especially useful in such a patient, given the criticality of blood pressure control and the more aggressive SBP and DBP goals (per JNC 7, <130 mmHg and <80 mmHg respectively) in such a patient. The patient can be, but is not necessarily, one exhibiting resistance to a baseline antihypertensive therapy, for example a patient having medically diagnosed resistant hypertension. The darusentan can be delivered by any suitable route of administration, typically orally, for example at a dosage amount and frequency as described above. Darusentan monotherapy or combination or adjunctive therapy as described herein can be administered.

In a still further embodiment, a method for lowering blood pressure in a patient exhibiting a bimodal waveform diurnal ABP pattern comprises administering darusentan to the patient. Again, the patient can be, but is not necessarily, one exhibiting resistance to a baseline antihypertensive therapy, for example a patient having medically diagnosed resistant hypertension. The darusentan can be delivered by any suitable route of administration, typically orally, for example at a dosage amount and frequency as described above. Darusentan monotherapy or combination or adjunctive therapy as described herein can be administered. In various aspects according to this embodiment, the 24-hour systolic and/or diastolic ABP is lowered, the maximum diurnal systolic and/or diastolic ABP is lowered, and/or the day/night ABP ratio is increased by practice of the method.

In a still further embodiment, a method for preventing one or more cardiovascular adverse events in a patient having medically diagnosed resistant hypertension comprises administering darusentan to the patient. Examples of cardiovascular adverse effects include without limitation acute coronary syndrome (including unstable angina and non-Q wave infarction), myocardial infarction, heart failure, systolic heart failure, diastolic heart failure (also known as diastolic dysfunction), stroke, occlusive stroke, hemorrhagic stroke and combinations thereof. “Preventing” in the present context includes reducing risk, incidence and/or severity of a subsequent cardiovascular adverse effect. Again, the darusentan can be delivered by any suitable route of administration, typically orally, for example at a dosage amount and frequency as described above. Darusentan monotherapy or combination or adjunctive therapy as described herein can be administered.

In a still further embodiment, a method for providing a beneficial effect on renal function in a patient having medically diagnosed resistant hypertension comprises administering darusentan to the patient. “Providing a beneficial effect” in the present context includes enhancing, maintaining or moderating a decline in renal function. Again, the darusentan can be delivered by any suitable route of administration, typically orally, for example at a dosage amount and frequency as described above. Darusentan monotherapy or combination or adjunctive therapy as described herein can be administered.

A beneficial effect on renal function can be observed, for example, by monitoring one or more blood and/or urinary biomarkers. Examples of such biomarkers include without limitation serum creatinine, serum insulin, serum glutamic acid decarboxylase (GAD), serum protein tyrosine phosphatase-like molecule IA2, blood urea nitrogen, urinary protein, urinary albumin, microalbuminuria, urinary β2-microglobulin, urinary N-acetyl-β-glucosaminidase, urinary retinol binding protein, urinary sodium, glomerular filtration rate, urinary albumin to creatinine ratio, urine volume, and combinations thereof.

Illustratively, the darusentan can be administered in a dose effective to lower urinary albumin to creatinine ratio. This can be especially beneficial where the baseline urinary albumin to creatinine ratio is greater than about 30 mg/g or where baseline 24-hour urinary albumin is greater than about 30 mg/day.

### EXAMPLES

The following examples are merely illustrative, and do not limit this disclosure in any way. Reference is made in the examples to statistical analysis and statistical significance of results. Such reference is made in the interest of full disclosure and does not constitute admission that statistical significance is a prerequisite for patentability of any claim herein.

### Example 1

### Summary

Despite treatment with and adherence to a diuretic plus multiple concomitant antihypertensives at full doses, blood pressure control remains suboptimal in a substantial number of patients with hypertension.

In this randomized, double-blind, placebo-controlled, dose-titration study, 115 patients with resistant hypertension as defined by JNC 7 guidelines were randomized 2:1 to receive escalating doses of darusentan (10, 50, 100, 150 and 300 mg) or placebo once daily for 10 weeks. Darusentan dose was increased every 2 weeks.

Darusentan decreased placebo-adjusted mean SBP and DBP from baseline to week 10 (−11.6 and −5.8 mmHg respectively; p<0.05). By the end of treatment, average trough sitting blood pressure decreased from 146.3/80.6 mmHg at baseline to 132.6/73.9 mmHg in patients treated with darusentan. Darusentan provided 24-hour blood pressure lowering benefits, as evidenced by decreases in 24-hour and nocturnal blood pressures measured by ambulatory blood pressure monitoring. Darusentan was generally well tolerated; mild to moderate edema was the most common adverse event. There were no clinically relevant effects on heart rate or liver enzyme levels.

It is concluded that darusentan has the potential to provide additional blood pressure lowering benefits when administered as an add-on therapy in patients with hypertension who are refractory to treatment with a diuretic plus two or more antihypertensive medications. This is the first study to show a clinical benefit from a new class of antihypertensive in patients who are classified as resistant by JNC 7 guidelines.

### Methods

### Patients

Patients (35 to 85 years of age) with resistant hypertension as defined by the JNC 7 guidelines and who were treated with and adhered to full doses of JNC 7 recommended antihypertensives, including a diuretic and two or more antihypertensive medications from different drug classes, defined as (a) ACE inhibitors and angiotensin II receptor blockers, (b) beta-adrenergic receptor blockers, and (c) calcium channel blockers, were eligible to participate in the study. Patients also were required to have an estimated glomerular filtration rate (GFR)≧30 mL/min/1.73 m² during the screening period. Women of childbearing potential were required to have a negative serum pregnancy test at the screening visit and a negative urine pregnancy test at baseline, and they must have agreed to use a reliable double-barrier method of contraception throughout the study and for at least 4 weeks after the last study visit. Patients with an average sitting SBP≧180 mmHg, DBP≧110 mmHg, hemoglobin A1c>10%, hemoglobin concentration<10 g/dL, hematocrit<30%, serum thyroid stimulating hormone concentration>1.5× the upper limit of normal (ULN), or serum alanine aminotransferase (ALT) or aspartate aminotransferase (AST)>2×ULN during the screening period were excluded from the study. Patients who had symptomatic arrhythmias, unstable angina pectoris, symptomatic congestive heart failure or hemodynamically significant valvular heart disease; had significant pulmonary disease; had experienced a myocardial infarction, unstable angina, or a cerebrovascular accident within 6 months of screening; were undergoing hemodialysis or peritoneal dialysis or had a history of renal transplant; or were receiving prohibited medications were also ineligible to participate in the study. All patients provided written informed consent.

### Study Design and Treatment

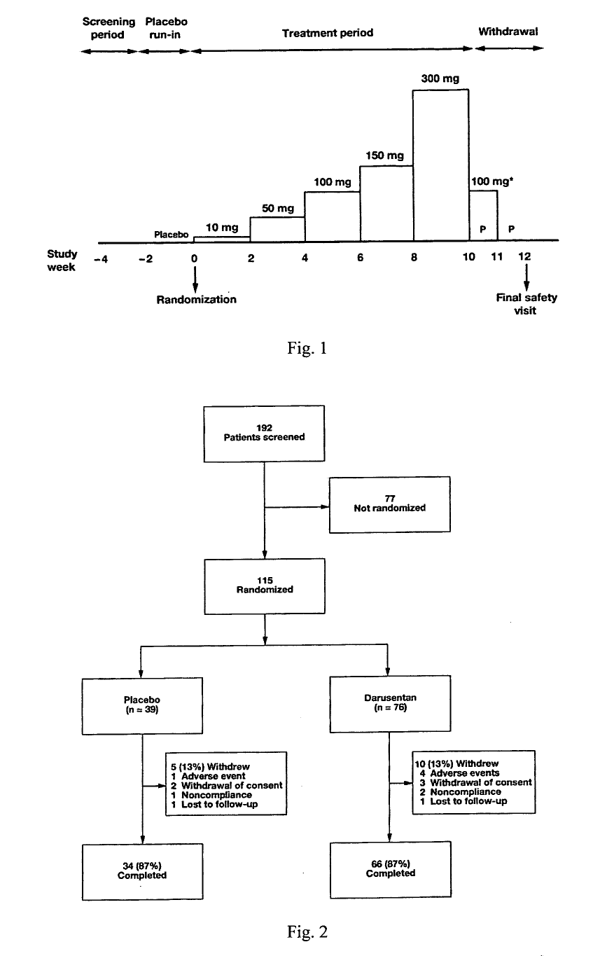

In this randomized, double-blind, placebo-controlled, dose-titration study, patients were randomized 2:1 to receive escalating doses of darusentan or placebo once daily in the morning for 10 weeks after completing a 2-week placebo run-in period (FIG. 1). Darusentan was initiated at a dose of 10 mg/day and escalated every 2 weeks at doses of 50, 100 and 150 mg/day until reaching a maximum of 300 mg/day. One blinded dose reduction was allowed in patients who did not tolerate an up-titration. Patients were withdrawn from the study if a subsequent dose reduction was required. After completion of the 10-week treatment period, patients were withdrawn from study medication over a maximum of 2 weeks. This study was approved by an Institutional Review Board/Independent Ethics Committee and conducted in accordance with the principles of Good Clinical Practices and the revised Declaration of Helsinki.

### Efficacy Assessments

Co-primary efficacy endpoints were the changes from baseline through weeks 8 and 10 (i.e., doses of 150 and 300 mg) in trough sitting SBP. Blood pressure was measured at every study visit (baseline and weeks 2, 4, 6, 8 and 10) using standard sphygmomanometry. Secondary variables included 24-hour SBP as measured by ambulatory blood pressure monitoring (ABPM), percentage of patients who achieved SBP goal, trough sitting DBP, and urinary albumin to creatinine ratio. Ambulatory blood pressure monitoring was recorded once before randomization and repeated during the 24 hours immediately preceding the week 10 study visit. Urine samples for determination of albumin to creatinine ratio were collected at screening, baseline and week 10.

### Safety Assessments

Physical examinations, vital sign measurements, clinical chemistry and hematologic laboratory tests, and electrocardiography were performed at the screening visit and periodically throughout the study. Blood samples were obtained at baseline and at weeks 4 and 10 during study drug treatment to monitor liver function. Additional blood samples were drawn from male patients at baseline and week 10 to assess follicle stimulating hormone, inhibin B, luteinizing hormone and testosterone levels. Male fertility was assessed by analysis of semen samples obtained within 2 weeks before baseline and within 2 weeks before week 10. Adverse events were monitored throughout the study.

### Statistical Methods

All patients who were randomized to treatment, received ≧1 dose of blinded study medication, and had a post-baseline blood pressure measurement were included in the efficacy analyses. A nonlinear mixed effect model was used for the analysis of change in blood pressure, with comorbidity status as a covariate. This model used all observed blood pressure measurements with no imputation for missing values. Linear contrast statements were used to test the slope through a given time point to test the effect of the dose administered on that week. Estimates of treatment effect were obtained via least square means. Response rates were compared with logistic regression models at each blood pressure measurement with comorbidity status as a covariate; missing values were imputed with last observation carried forward for these models. Safety summaries include all patients who received ≧1 dose of blinded study medication.

Type I error rate for analysis of multiple doses was controlled by using the Hommel method on the 2 co-primary time points (weeks 8 and 10, corresponding to the 150 and 300 mg doses). If ≧1 of these was significant, testing would proceed stepwise to week 6, week 4 and week 2 (doses of 100, 50 and 10 mg respectively) as long as the prior comparison was significant at α=0.05. This controlled the type I error rate for each measurement (SBP, DBP, response, etc.). In general, adjusted p values are reported herein unless it is explicitly noted that a given p value is nominal.

A sample size of 35 patients in the placebo arm and 70 patients in the darusentan arm was planned to provide at least 85% power to detect a difference from placebo for the darusentan 150 mg or 300 mg doses, assuming a placebo-adjusted reduction from baseline in trough sitting SBP of 8 mmHg, a standard deviation of 12 mmHg, and a correlation between week 8 and week 10 SBP change of 0.85.

### Results

### Patients

A total of 192 patients were screened and 115 patients were randomized; 76 received darusentan and 39 received placebo (FIG. 2). Patient demographics and baseline characteristics were generally similar between treatment groups (Table 1).

TABLE 1
Patient demographics and baseline characteristics

| | Darusentan | Placebo |
|---|---|---|
| Parameter | (n = 76) | (n = 39) |
| Age, years† | 62 ± 10 | 63 ± 11 |
| Age ≧65 years, n (%) | 33 (43) | 17 (44) |
| Sex, n (%) male | 43 (57) | 25 (64) |
| Race, n (%) | | |
| White | 53 (70) | 29 (74) |
| Black | 23 (30) | 9 (23) |
| American Indian or Alaska Native | 0 | 1 (3) |
| Body mass index, kg/m²† | 31.2 ± 5.0 | 32.6 ± 4.8 |
| Sitting SBP, mmHg† | 146.3 ± 13.3 | 147.1 ± 17.4 |
| Sitting DBP, mmHg† | 80.6 ± 12.3 | 78.7 ± 12.3 |
| Sitting pulse rate, beats/min† | 66.7 ± 10.6 | 68.7 ± 11.2 |
| Urinary albumin to creatinine ratio, | 94.5 ± 396.9 | 17.2 ± 18.0 |
| mg/g creatinine† | | |
| Estimated GFR, mL/min/1.73 m²† | 74.2 ± 24.3 | 79.3 ± 26.3 |
| Diabetes | 36 (47) | 19 (49) |
| Chronic kidney disease | 20 (26) | 9 (23) |
| Concomitant antihypertensives, n (%) | | |
| Diuretics | 76 (100) | 39 (100) |
| Calcium channel blockers | 56 (74) | 29 (74) |
| Angiotensin converting enzyme | 36 (47) | 22 (56) |
| inhibitors | | |
| Angiotensin II receptor blockers | 44 (58) | 20 (51) |
| Beta-blockers | 53 (70) | 25 (64) |
| Other | 15 (20) | 5 (13) |

GFR = glomerular filtration rate.
†Data are means ± standard deviations.

Apparent differences in baseline urinary albumin to creatinine ratio reflect a high degree of variability in this parameter in the darusentan group. Concomitant antihypertensive medication profiles were similar between treatment groups, with all patients receiving a diuretic and two or more other antihypertensives from different drug classes as specified above, all at full doses.

The majority of patients (87%) in each treatment group completed the study, with a mean treatment duration of 79 days for darusentan and 78 days for placebo. Most patients (78%) treated with darusentan were able to successfully escalate study medication dose to 300 mg/day. The most common reasons for study discontinuation were adverse events (5 patients), withdrawal of consent (5 patients), and lost to follow-up (3 patients) (FIG. 2).

### Efficacy of Darusentan

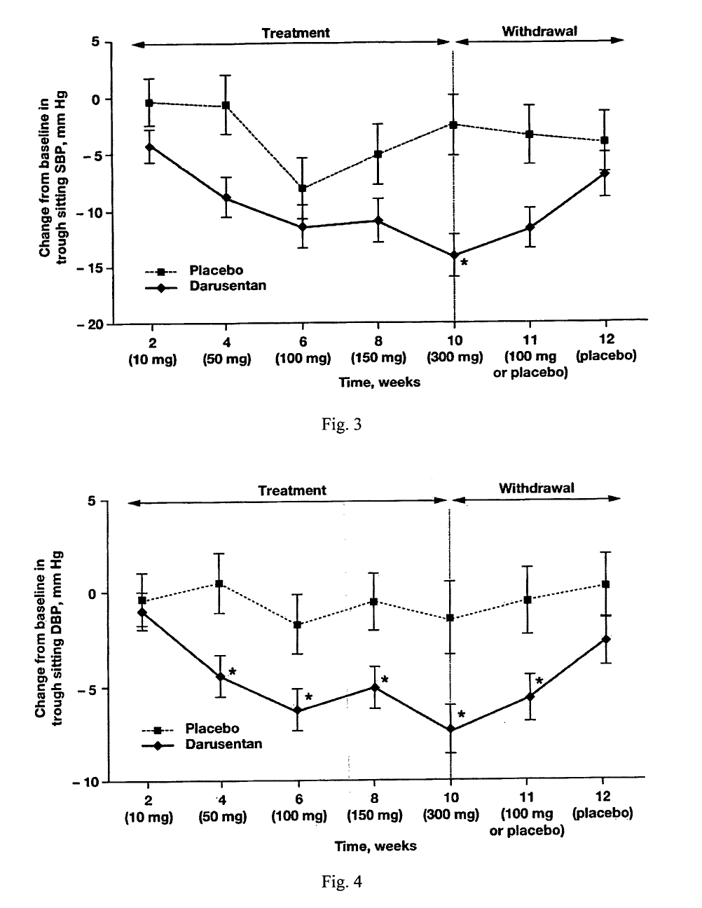

Darusentan significantly reduced placebo-adjusted mean trough sitting SBP after 10 weeks (300 mg dose) of treatment (−11.6 mmHg; p=0.02) (FIG. 3). Improvements in the co-primary variable, change from baseline to week 8 (150 mg dose) in mean trough sitting SBP, were evident at week 8, although the difference between groups was not statistically significant. In addition, by week 10 darusentan had reduced peak sitting SBP by 17.1 mmHg vs. 9.9 mmHg with placebo (p=0.023). At the end of treatment, 49% of patients in the darusentan group and only 28% of patients in the placebo group achieved SBP goal (p=0.068) (Table 2).

TABLE 2
Percent of patients achieving SBP goal

Responders, n (%)

| | Darusentan | Placebo | |
|---|---|---|---|
| Study week (dose) | (n = 76) | (n = 39) | p value† |
| Week 10 (300 mg) | 37 (48.7) | 11 (28.2) | 0.068 |
| Week 8 (150 mg) | 32 (42.1) | 12 (30.8) | 0.192 |
| Week 6 (100 mg) | 34 (44.7) | 17 (43.6) | 0.953 |
| Week 4 (50 mg) | 36 (47.4) | 9 (23.1) | 0.953 |
| Week 2 (10 mg) | 23 (30.3) | 7 (17.9) | 0.953 |

†Adjusted p values from the multiple comparisons procedure: Hochberg procedure for weeks 10 and 8, and fixed sequence procedure for weeks 2–6.

ABPM performed at week 10 revealed significant reductions in placebo-adjusted 24-hour SBP and DBP from baseline in patients treated with darusentan (−9.2 and −7.2 mmHg, respectively; p<0.001). Peak SBP and DBP, as recorded by ABPM, improved by 14.1 and 9.2 mmHg in the darusentan group and 6.0 and 2.3 mmHg in the placebo group respectively (p<0.05 between groups). Reductions in blood pressure were maintained throughout the 24-hour monitoring period. Darusentan significantly reduced mean ABPM-determined nocturnal blood pressure, with placebo-adjusted change from baseline values of −9.9 and −5.9 mm Hg for SBP and DBP, respectively, at week 10 (p<0.01 vs. placebo). Moreover, a post-hoc analysis demonstrated that darusentan improved placebo-adjusted diurnal SBP by 8.7 mmHg after 10 weeks of treatment.

Darusentan significantly reduced placebo-adjusted mean trough sitting DBP after 10 weeks of treatment (−5.8 mmHg; p=0.004). Significant improvements versus placebo were evident beginning at week 4 (−4.9 mmHg; p=0.01) and were maintained or further improved throughout the study (FIG. 3). By week 10, darusentan had reduced peak sitting DBP by 8.2 mmHg (p<0.01).

For both trough sitting SBP and DBP, improvements were dose dependent in patients treated with darusentan (FIG. 3). At study end, the average trough sitting blood pressures were 132.6/73.9 and 145.1/77.9 mmHg for the darusentan and placebo groups respectively. During the 2-week withdrawal period, sitting and standing blood pressure increased, although there was no significant difference between darusentan and placebo treatment groups.

### Darusentan Safety and Tolerability

Adverse events were generally mild to moderate in intensity. A total of 242 treatment-emergent adverse events were experienced by 81 patients over the course of the study (Table 3).

TABLE 3
Treatment-emergent adverse events*

Patients, n (%)

| | Darusentan | Placebo |
|---|---|---|
| Adverse event | (n = 76) | (n = 39) |
| Peripheral edema | 13 (17) | 2 (5) |
| Headache | 8 (11) | 2 (5) |
| Sinusitis | 6 (8) | 0 |
| Dizziness | 5 (7) | 1 (3) |
| Nasopharyngitis | 5 (7) | 1 (3) |
| Upper respiratory tract infection | 4 (5) | 2 (5) |
| Gastroenteritis | 4 (5) | 1 (3) |
| Arthralgia | 2 (3) | 4 (10) |
| Diarrhea | 1 (1) | 2 (5) |
| Arthritis | 0 | 2 (5) |

*Occurring in ≧5% of patients in a treatment group.

The most common adverse events among patients in the darusentan group, namely peripheral edema (17% of patients) and headache (11% of patients), were consistent with the mechanism of action and pharmacodynamic effects of endothelin receptor antagonists. Peripheral edema was largely mild to moderate in intensity, with one case of severe edema reported. Five serious adverse events were reported by 4 patients in the darusentan group (coronary artery disease, aseptic meningitis, pneumonia, lung squamous cell carcinoma, and pleural effusion), and one serious adverse event was reported by a patient in the placebo group (ischemic colitis). None of these events were considered to be related to study medication. Only one serious adverse event (pleural effusion) led to study discontinuation. A total of 4 patients in the darusentan group and 1 patient in the placebo group discontinued the study because of an adverse event. No change in frequency or severity of adverse events over time was observed. No patients died during the course of the study.

Heart rate was not affected by treatment with darusentan, with a change from baseline of 0.4±0.9 beats per minute at week 10, which was comparable with the change of 2.3±1.3 beats per minute observed in the placebo group.

The geometric mean urinary albumin to creatinine ratio increased slightly (6.2%) over baseline in the placebo group but decreased by 25.3% from baseline in the darusentan group, indicative of a possible beneficial effect of darusentan on renal function. The reduction in geometric mean urinary albumin to creatinine ratio was especially pronounced in those patients having a baseline albumin to creatinine ratio>30 mg/g (Table 4).

TABLE 4
Albumin to creatinine ratio changes from baseline

| | | |
|---|---|---|
| | Geometric mean | |
| | albumin to creatinine | % Change |
| Treatment | ratio (mg/g) | from baseline |

| | | | | |
|---|---|---|---|---|
| group | n | Baseline | Week 10 | Mean (SE) |

All observed patients

| | | | | |
|---|---|---|---|---|
| Darusentan | 65 | 12.6 | 9.4 | −25.3 (8.9) |
| Placebo | 34 | 9.1 | 9.7 | +6.2 (12.2) |

Baseline ratio ≦30 mg/g

| | | | | |
|---|---|---|---|---|
| Darusentan | 53 | 7.2 | 5.9 | −18.4 (8.3) |
| Placebo | 28 | 6.4 | 6.8 | +5.2 (10.6) |

Baseline ratio >30 mg/g

| | | | | |
|---|---|---|---|---|
| Darusentan | 12 | 145.4 | 73.6 | −49.4 (23.6) |
| Placebo | 6 | 46.5 | 51.5 | +10.8 (53.7) |

Other relevant parameters, including male hormone level changes, were similar between treatment groups. Decreases in hematocrit and hemoglobin reported as adverse events each occurred in 2 patients treated with darusentan; all events were mild in severity. Liver function test results were comparable between treatment groups; mean concentrations of ALT, AST, and γ-glutamyltransferase decreased slightly from baseline in both groups. Furthermore, no patients experienced elevations in ALT or AST>2×ULN.

### Discussion

Darusentan up-titrated from 10 to 300 mg once daily over 10 weeks achieved clinically meaningful improvements in sitting trough SBP and DBP in a difficult-to-treat resistant hypertension patient population, namely patients with uncontrolled blood pressure despite stringent adherence to an appropriate regimen of three or more drugs, including a diuretic. Reductions in SBP and DBP were dose dependent, with the greatest benefit observed after 2 weeks of treatment with darusentan 300 mg/day (i.e., week 10). Conclusions about the effect of lower doses of darusentan are complicated by a study design that confounded time and dose; however, there is evidence that a dose at least as low as 10 mg can provide a clinically important decrease in both SBP and DBP. Since the effect of 10 mg of darusentan is a robust decrease in systolic blood pressure, it is highly likely that lower doses of darusentan will also decrease systolic blood pressure in some patients. Moreover, the effect of darusentan on blood pressure was not accompanied by changes in heart rate, as changes in heart rate were minimal and comparable with placebo.

For the co-primary variables, namely change from baseline in sitting trough SBP at weeks 8 and 10, reductions with darusentan treatment were statistically significant at week 10. The placebo effect was quite substantial during weeks 6 and 8 (−8.3 and −5.1 mmHg) respectively, thereby limiting ability to detect significant improvements over those of placebo in the darusentan group. A large placebo effect has been reported for other hypertension trials. However, the placebo effect is not as evident in ABPM.

ABPM is an effective tool to examine the 24-hour profile of blood pressure fluctuations, allowing for determination of daytime and nighttime blood pressure lowering effects of study medication. Standard cuff measurements performed in the office may overestimate blood pressure if they are not performed correctly or if an inappropriate cuff size is used. Furthermore, ABPM also helps to distinguish true drug-resistant hypertension from white-coat hypertension (elevation of blood pressure limited to clinic/office visits).

The blood pressure lowering benefits of darusentan were maintained over 24 hours; both 24-hour ABPM and nocturnal ABPM measurements were significantly reduced relative to placebo by the end of the study. Maintained efficacy throughout the day supports a convenient once daily dosing regimen for darusentan. A once daily dosing schedule is consistent with many currently available antihypertensives, allowing for coordination of drug administration in a patient population already challenged with a high pill burden (polypharmacy).

By strictly adhering to the JNC 7 guidelines, this study was unique in that it enrolled a clearly defined patient population and limited contributing factors that may mimic resistant hypertension. Neither the World Health Organization/International Society of Hypertension (WHO/ISH) nor British Hypertension Society (BHS) guidelines define resistant hypertension, whereas the previous version of the JNC guidelines (JNC 6) recommended less stringent criteria for resistant hypertension, with a higher blood pressure goal in older patients with isolated systolic hypertension (SBP<160 mmHg). In the 2003 European Society for Hypertension/European Society of Cardiology (ESH/ESC) guidelines, the use of a diuretic is not included in the definition of resistant hypertension. The inclusion of diuretics in the JNC 7 definition of resistant hypertension is an important distinction because the use of diuretics limits the potential for resistance attributable to volume overload.

The population in this study was largely composed of patients with chronic kidney disease or diabetes. Diabetes and hypertension are co-risk factors; the prevalence of hypertension is significantly higher among diabetics than in the general population, and vice versa. Risk of cardiovascular disease is greatly increased among patients with chronic kidney disease or diabetes, thus adding an additional risk over that of hypertension alone. In an effort to reduce cardiovascular disease risk and progression of diabetic nephropathy to end-stage renal disease, the target blood pressure goal in JNC 7 was reduced from 140/90 mmHg to 130/80 mmHg in these patient populations. Lowering blood pressure targets among already difficult-to-treat patients is likely to expand the proportion of patients who will be classified as resistant.

Darusentan was generally safe and well tolerated in this patient population. The majority of adverse events were mild to moderate, with few serious adverse events or patients who withdrew from the study because of adverse events. The severity of peripheral edema (a class effect of endothelin receptor antagonists) was mild to moderate and infrequently resulted in discontinuation. It is important to note that in this study no changes in diuretic regimen were allowed because of edema, which may overestimate the incidence of edema expected in the clinical setting. A significant safety finding was the lack of liver function test abnormalities that are commonly associated with daily treatment with endothelin receptor antagonists. Indeed, no patients experienced elevations in ALT or AST>2×ULN.

A limitation of this study was the lack of independent dose groups. Patients escalated dose based on 2-week intervals, limiting ability to assess efficacy of a single darusentan dosage over time. Long-term studies with discrete dose groups would be necessary to fully evaluate an appropriate darusentan dosage for patients with resistant hypertension.

In conclusion, darusentan appears to provide additional blood pressure lowering benefit as an add-on antihypertensive therapy in patients with resistant hypertension receiving three or more antihypertensive therapies, including a diuretic and at least two of (a) an ACE inhibitor or angiotensin II receptor blocker, (b) a beta-adrenergic receptor blocker and (c) a calcium channel blocker. Moreover, the safety and tolerability profile of darusentan was favorable; most patients tolerated the maximum administered dose of darusentan.

### Example 2

Based on the study described in Example 1, the following additional statements are made.

Resistant hypertension is defined by JNC 7 as the failure to achieve goal blood pressure in patients who are adhering to full doses of an appropriate three-drug antihypertensive regimen that includes a diuretic. Darusentan is a selective ET_{A} receptor antagonist, and an objective of the present study is to examine whether darusentan may provide antihypertensive effects via a mechanism of action independent of other classes of antihypertensive drugs when used as adjunctive therapy in patients with resistant hypertension.

Subjects with resistant hypertension, adhering to a regimen of at least three antihypertensives including a diuretic at documented full doses, were randomized 2:1 to blinded oral darusentan or placebo once daily. Following a 2-week placebo run-in, subjects underwent forced-titration of study drug every 2 weeks through doses of 10, 50, 100, 150 and 300 mg (10 weeks total). The co-primary endpoints were change from baseline in trough sitting SBP to week 8 and to week 10 (150 and 300 mg doses, respectively, with adjustment for comparison of two doses) as compared to placebo. Secondary endpoints were change from baseline in trough sitting DBP, mean 24-hour ambulatory SBP, and percent of subjects reaching JNC 7 SBP goals.

Of the 115 randomized subjects, 61% had diabetes and/or chronic kidney disease and 76 received darusentan. At baseline, mean SBP was 146.6±14.8 mmHg and mean DBP was 80.0±12.3 mmHg. At week 10 of darusentan treatment, the placebo-corrected change from baseline in SBP was −11.6±3.3 mmHg (p=0.02) and −5.8±2.3 mmHg (p=0.004) for DBP. Changes in mean ambulatory 24-hour placebo-corrected SBP and DBP were −9.2±2.2 mmHg (p<0.001) and −7.2±1.6 mmHg (p<0.001) respectively. In addition, 49% of darusentan-treated versus 28% of placebo-treated subjects (p=0.068) achieved JNC 7 SBP goals. Darusentan was well tolerated with no drug-related serious adverse events or elevations in serum aminotransferase concentrations>2×ULN. The most frequent adverse event was peripheral edema (17%). There were 15 premature discontinuations (10 darusentan).

Darusentan produced clinically and statistically significant reductions in SBP and DBP in subjects with resistant hypertension who were adhering to documented full doses of at least three antihypertensive drugs.

### Example 3

Based on the study described in Example 1, the following additional statements are made.

Darusentan is an ET_{A}-selective endothelin receptor antagonist that has now been demonstrated to produce clinically and statistically significant reductions in trough sitting blood pressure, as measured by standard sphygmomanometry, in 115 patients with resistant hypertension receiving documented full doses of at least three antihypertensive drugs, including a diuretic. In this randomized, double-blind, multi-center study, subjects underwent a 2-week placebo run-in, followed by 2:1 randomization to darusentan or placebo once daily for 10 weeks. Subjects were initiated on 10 mg of study drug and underwent dose-escalation every 2 weeks through doses of 50, 100 and 150 mg until a maximum dose of 300 mg was achieved. Change from baseline to week 10 in 24-hour ambulatory blood pressure (ABP) was a secondary efficacy endpoint. All available ABP data were included in the analyses (observed population).

Treatment with 300 mg darusentan resulted in significant placebo-adjusted reductions in mean 24-hour systolic and diastolic ABP of 9.2=2.2 mmHg (p<0.001) and 7.2±1.6 mmHg (p<0.001) respectively. At week 10, day-time SBP decreased by 10.9±1.4 mmHg in the darusentan group and 2.2±2.4 mmHg in the placebo group (p<0.001), and night-time SBP decreased by 11.9±1.6 mmHg on darusentan vs. 3.8±2.7 mmHg on placebo (p=0.004). Day/night SBP ratio at week 10 was 9.5% on darusentan vs. 6.8% on placebo (p=0.051). An evaluation of mean hourly systolic ABP over the 24-hour monitoring period indicated that blood pressure remained consistently lower throughout the dosing interval in subjects treated with darusentan as compared to baseline or placebo (FIG. 5).

Darusentan administered once daily resulted in significant and sustained reductions in ABP when used in combination with at least three antihypertensive drugs in patients with resistant hypertension.

### Example 4

Based on the study described in Example 1, the following additional statements are made.

Many patients with resistant hypertension do not achieve guideline-recommended SBP goals despite treatment with multi-drug antihypertensive regimens. Darusentan is an ET_{A}-selective endothelin receptor antagonist that has now been demonstrated to significantly decrease trough cuff SBP and 24-hour ambulatory SBP in patients with RHTN receiving full doses of 3 or more antihypertensive drugs.

A randomized, double-blind, placebo-controlled, multi-center, dose-ranging study of oral darusentan was conducted in 115 patients with resistant hypertension as defined by JNC 7 guidelines. Following a 2-week placebo run-in, eligible subjects were randomized 2:1 to darusentan or placebo for 10 weeks. Study drug was initiated at 10 mg/day and the dose was increased every 2 weeks, through doses of 50, 100 and 150 mg, until a maximum dose of 300 mg was achieved. Percent of subjects meeting SBP goal, as defined by JNC 7 guidelines, was a pre-specified secondary endpoint in the study. Additional analyses to examine percent of subjects responding to darusentan therapy were performed post-hoc. Probability (p) values were unadjusted for multiple comparisons.

The percent of subjects responding to darusentan therapy, defined as achieving SBP goal or a decrease in SBP of at least 10 mmHg as compared to baseline values, was 71% at week 10 versus 49% in the placebo group (p=0.021). A change from baseline to week 10 of at least 10 mmHg for SBP was reported in approximately 64% of subjects on darusentan as compared to 46% on placebo (p=0.064). Furthermore, about 46% of darusentan subjects experienced a decrease in SBP of at least 20 mmHg as compared to 26% of placebo subjects (p=0.032).

It is concluded that darusentan reduces SBP and allows more resistant hypertension patients to achieve guideline-recommended SBP goals when added to a regimen that includes at least three full-dose antihypertensive drugs.

### Example 5

Based on the study described in Example 1, the following additional statements are made.

Patients with resistant hypertension do not achieve guideline-recommended SBP goals despite treatment with multi-drug antihypertensive regimens.

A randomized, double-blind, placebo-controlled, multi-center, dose-ranging study of oral darusentan was performed in 115 patients with resistant hypertension. Resistant hypertension was defined by JNC 7 guidelines and subjects were on full doses of three or more antihypertensive drugs including a diuretic. Following a 2-week placebo run-in, eligible subjects were randomized 2:1 to darusentan or placebo for 10 weeks. Darusentan was initiated at 10 mg/day and the dose was up-titrated every 2 weeks, through doses of 50, 100 and 150 mg, until a maximum dose of 300 mg was achieved. The primary endpoint of the study examined change from baseline in trough sitting SBP. Secondary endpoints included change from baseline in DBP, 24-hour ambulatory SBP, and percent of subjects achieving guideline-recommended SBP goal. Subgroup analyses to evaluate the effects of darusentan according to the number of antihypertensive medications received at baseline were performed post-hoc. Probability (p) values were unadjusted for multiple comparisons.

Sixty-four subjects were on exactly 3 antihypertensive medications and 51 subjects were on 4 or more antihypertensive drugs at baseline. At week 10 of darusentan treatment (300 mg dose), the placebo-corrected change from baseline in trough sitting SBP was −9.39 mmHg (p=0.03) in the subjects on exactly 3 antihypertensive drugs, by comparison with −12.71 mmHg (p=0.007) for subjects on ≧4 antihypertensive drugs. Although not powered to demonstrate statistical significance, the percent of subjects achieving SBP goal or a decrease in SBP of at least 10 mmHg, as compared to baseline values, was 76% on darusentan at week 10 vs. 57% in the placebo group (p=0.11) in the subjects on exactly 3 antihypertensive drugs, and was 66% vs. 38% on placebo (p=0.06) in the subjects on ≧4 antihypertensive drugs.

The present study shows that darusentan significantly reduces SBP and may allow more resistant hypertension patients to achieve guideline-recommended SBP goals when added to a regimen that includes 3, 4 or more antihypertensive drugs. Darusentan may be useful for reducing SBP in patients with resistant hypertension independent of the number of background antihypertensive drugs.

All patents and publications cited herein are incorporated by reference into this application in their entirety.

The words “comprise”, “comprises”, and “comprising” are to be interpreted inclusively rather than exclusively.

What is claimed is:

1. A method for lowering blood pressure in a patient having clinically diagnosed resistant hypertension, the method comprising administering darusentan to the patient adjunctively with a baseline antihypertensive regimen that comprises administration of at least one diuretic and at least two antihypertensive drugs selected from at least two of (a) ACE inhibitors and angiotensin II receptor blockers, (b) beta-adrenergic receptor blockers and (c) calcium channel blockers; wherein the darusentan is orally administered at a dose and frequency effective, in combination with said regimen, to provide a reduction of at least about 3 mmHg in one or more blood pressure parameters selected from trough sitting systolic, trough sitting diastolic, 24-hour ambulatory systolic, 24-hour ambulatory diastolic, maximum diurnal systolic and maximum diurnal diastolic blood pressures.

2. The method of claim 1, wherein the patient has clinically diagnosed resistant systolic hypertension, and the dose and frequency of darusentan administration is effective in combination with the baseline regimen to provide a reduction of at least about 3 mmHg in one or more systolic blood pressure parameters selected from trough sitting, 24-hour ambulatory and maximum diurnal systolic blood pressures.

3. The method of claim 2, wherein a JNC 7 goal for systolic blood pressure is achieved.

4. The method of claim 1, wherein the patient has clinically diagnosed resistant diastolic hypertension, and the dose and frequency of darusentan administration is effective in combination with the baseline therapy to provide a reduction of at least about 3 mmHg in one or more diastolic blood pressure parameters selected from trough sitting, 24-hour ambulatory and maximum diurnal diastolic blood pressures.

5. The method of claim 4, wherein a JNC 7 goal for diastolic blood pressure is achieved.

6. The method of claim 1, wherein an increase in day/night ambulatory blood pressure ratio is provided.

7. The method of claim 1, wherein the patient has medically diagnosed diabetes, chronic kidney disease or both.

8. The method of claim 1, wherein the patient at baseline exhibits a bimodal waveform diurnal pattern of ambulatory systolic or diastolic blood pressure.

9. The method of claim 1, wherein the darusentan is administered adjunctively with at least one diuretic selected from the group consisting of acetazolamide, althiazide, ambuside, amiloride, aminometradine, azosemide, bendroflumethiazide, benzthiazide, benzylhydrochlorothiazide, Biogen BG 9719, bumetanide, butazolamide, buthiazide, canrenone, chloraminophenamide, chlorazanil, chlormerodrin, chlorothiazide, chlorthalidone, clofenamide, clopamide, clorexolone, cyclopenthiazide, cyclothiazide, disulfamide, ethacrynic acid, ethiazide, ethoxzolamide, etozolin, fenquizone, furosemide, hydrochlorothiazide, hydroflumethiazide, indapamide, isosorbide, Kiowa Hakko KW 3902, mannitol, mefruside, meralluride, mercaptomerin sodium, mercumatilin sodium, mercurous chloride, mersalyl, methazolamide, methyclothiazide, metolazone, muzolimine, oleandrin, pamabrom, paraflutizide, perhexiline, piretanide, polythiazide, protheobromine, quinethazone, spironolactone, Sanofi-Aventis SR 121463, teclothiazide, theobromine, ticrynafen, torsemide, triamterene, trichlormethiazide, tripamide, urea, xipamide and combinations thereof.

10. The method of claim 1, wherein the darusentan is administered adjunctively with at least one ACE inhibitor selected from the group consisting of alacepril, benazepril, captopril, ceronapril, cilazapril, delapril, enalapril, enalaprilat, eosinopril, fosinopril, imidapril, lisinopril, moexipril, moveltipril, omapatrilat, perindopril, quinapril, ramipril, sampatrilat, spirapril, temocapril, trandolapril and combinations thereof.

11. The method of claim 1, wherein the darusentan is administered adjunctively with at least one angiotensin II receptor blocker selected from the group consisting of candesartan, eprosartan, irbesartan, losartan, olmesartan, tasosartan, telmisartan, valsartan and combinations thereof.

12. The method of claim 1, wherein the darusentan is administered adjunctively with at least one beta-adrenergic receptor blocker selected from the group consisting of AC 623, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butidrine hydrocholoride, butofilolol, carazolol, carteolol, carvedilol, celiprolol, cetamolol, cloranolol, dilevalol, esmolol, indenolol, labetalol, landiolol, levobunolol, mepindolol, metipranolol, metoprolol, moprolol, nadolol, nadoxolol, nebivolol, nifenalol, nipradilol, oxprenolol, penbutolol, pindolol, practolol, pronethalol, propranolol, sotalol, sulfinalol, talinolol, tertatolol, tilisolol, timolol, toliprolol, xibenolol and combinations thereof.

13. The method of claim 1, wherein the darusentan is administered adjunctively with at least one calcium channel blocker selected from the group consisting of amlodipine, aranidipine, barnidipine, bencyclane, benidipine, bepridil, cilnidipine, cinnarizine, clentiazem, diltiazem, dotarizine, efonidipine, elgodipine, etafenone, fantofarone, felodipine, fendiline, flunarizine, gallopamil, isradipine, lacidipine, lercanidipine, lidoflazine, lomerizine, manidipine, mibefradil, monatepil, nicardipine, nifedipine, nilvadipine, nimodipine, nisoldipine, nitrendipine, NZ 105, perhexiline, prenylamine, semotiadil, terodiline, verapamil and combinations thereof.

14. The method of claim 1, wherein the darusentan is administered adjunctively with
(a) a diuretic selected from the group consisting of chlorothiazide, chlorthalidone, hydrochlorothiazide, indapamide, metolazone, polythiazide, bumetanide, furosemide, torsemide and combinations thereof; and at least two of
(b) an angiotensin converting enzyme inhibitor selected from the group consisting of benazepril, captopril, enalapril, fosinopril, lisinopril, moexipril, perindopril, quinapril, ramipril, trandolapril and combinations thereof, and/or an angiotensin II receptor blocker selected from the group consisting of candesartan, eprosartan, irbesartan, losartan, olmesartan, tasosartan, telmisartan, valsartan and combinations thereof;
(c) a beta-adrenergic receptor blocker selected from the group consisting of acebutolol, atenolol, betaxolol, bisoprolol, carvedilol, labetalol, metoprolol, nadolol, penbutolol, pindolol, propranolol, timolol and combinations thereof; and
(d) a calcium channel blocker selected from the group consisting of amlodipine, diltiazem, felodipine, isradipine, nicardipine, nifedipine, nisoldipine, verapamil and combinations thereof.

15. The method of claim 1, wherein the dose of darusentan is about 1 to about 600 mg/day.

16. The method of claim 1, wherein the dose of darusentan is about 10 to about 300 mg/day.

17. The method of claim 1, wherein the frequency of administration of darusentan is once a day.

18. The method of claim 17, wherein the darusentan is administered adjunctively with at least one diuretic or antihypertensive drug that is also orally administered once a day.

19. The method of claim 18, wherein the darusentan is coformulated in a fixed dose combination with said at least one diuretic or antihypertensive drug.

20. The method of claim 1, wherein darusentan and all drugs administered adjunctively therewith are orally administered once a day at or about the same time each day.
